(19) United States
(12) Patent Application Publication
(10) Pub. No.: US 2017/0165183 A1
(43) Pub. Date: Jun. 15, 2017

(54) AGENT AND METHOD FOR THE TEMPORARY DEFORMATION OF KERATIN-CONTAINING FIBERS

(71) Applicant: Henkel AG & Co. KGaA, Duesseldorf (DE)

(72) Inventors: Thorsten Knappe, Schenefeld (DE); Pamela Kaftan, Hamburg (DE); Maria Catalina Bermudez Agudelo, Hamburg (DE); Tim Bethge, Hamburg (DE)

(73) Assignee: Henkel AG & Co. KGaA, Duesseldorf (DE)

(21) Appl. No.: 15/365,139

(22) Filed: Nov. 30, 2016

(30) Foreign Application Priority Data
Dec. 15, 2015 (DE) 10 2015 225 204.3

(51) Int. Cl. A61K 8/81; A61K 8/31; A61Q 5/06; A61K 8/34

(52) U.S. Cl. CPC A61K 8/8158; A61K 8/34; A61K 8/8147; A61K 8/315; A61K 8/31; A61Q 5/06; A61K 2800/594

(57) ABSTRACT

Cosmetic agents and methods for the temporary deformation of keratinous fibers using the cosmetic agents are provided herein. In one embodiment, the cosmetic agent for the temporary deformation of keratinous fibe includes a cosmetic preparation. The cosmetic preparation contains at least one copolymer a1) composed at least of the following monomer units: styrene, and at least one of acrylic acid or methacrylic acid. The cosmetic preparation further contains at least one copolymer a2) composed at least of the following monomer units: N-tert-butylacrylamide, acrylic acid, and ethyl acrylate. The weight fraction of copolymers a1) and a2) in the total weight of the cosmetic preparation is about 10 to about 30% by weight.

### CROSS-REFERENCE TO RELATED APPLICATION

This application claims priority to German Patent Application No. 10 2015 225 204.3, filed Dec. 15, 2015, which is incorporated herein by reference in its entirety.

### TECHNICAL FIELD

This disclosure relates to a cosmetic composition for setting hair or for temporarily deforming keratinous fibers, in particular human hair, wherein the composition contains a combination of two specific copolymers.

### BACKGROUND

The temporary shaping of hairstyles for an extended period of time of up to several days generally requires the use of setting active substances. For this reason, hair treatment agents which are used to temporarily shape the hair play an important role. Corresponding agents for the temporary deformation usually contain synthetic polymers and/or waxes as the setting active substance. Agents for assisting in the temporary deformation of keratin-containing fibers may be provided as hair spray, hair wax, hair gel, or hair foam, for example.

The most important property of an agent for the temporary deformation of hair, also referred to below as a styling agent, is to provide the treated fibers with a preferably strong hold in the reshaped form, i.e., a shape that is imparted to the hair. This is also referred to as a strong hairstyle hold or a high degree of hold of the styling agent. The hairstyle hold is determined essentially by the type and quantity of the setting active substances used, although influence by the other components of the styling agent may also be provided.

Styling agents must meet numerous other requirements besides a high degree of hold. These may be roughly divided into properties on the hair, properties of the particular formulation, such as properties of sprayed aerosols, and properties relating to the use of the styling agent, particular importance being attached to the properties on the hair. Mentioned in particular are moisture resistance, low tack, and a balanced conditioning effect. In addition, a styling agent should preferably be universally usable for all types of hair and be mild on the hair and skin.

In order to meet the various requirements, a number of synthetic polymers used in styling agents have already been developed as setting active substances. These polymers may be divided into cationic, anionic, nonionic, and amphoteric setting polymers.

Hair-setting agents based on copolymers of styrene with (meth)acrylic acid and/or the esters thereof are described in International Patent application WO 2012/168035 A1.

Hair sprays based on copolymers of N-tert-butylacrylamide, acrylic acid, and ethyl acrylate are described in German Patent application DE 10 2004 040 172 A1, among other publications.

Not every polymer, and not every polymer mixture, is basically suited for producing hair styling agents. This is particularly true for hair sprays, in which, for example, the viscosity and thus also the spray characteristics are influenced by the polymer and the quantity of polymer used.

In addition, not every polymer, and not every polymer mixture, is suited for producing highly concentrated hair styling agents. With increasing polymer concentration, in addition to the problems with the viscosity and dosability of the agents described above, difficulties also occur with regard to the solubility and the storage stability, in particular of hair sprays.

A hair spray having a high polymer content (compact hair spray) is described in International Patent application WO 2005/012588 A2, for example.

Although suitable polymers and polymer combinations have been developed some time ago for use in styling concentrates in the area of temporary hair deformation, the results achieved thus far still leave room for improvements, in particular with regard to the storage stability, the applicability, and the degree of hold of these agents. In particular, currently available styling agents are in need of further improvement, since a good combination of the degree of hold and the long-term hold (high humidity curl retention) is not always sufficiently ensured.

### BRIEF SUMMARY

Cosmetic agents and methods for the temporary deformation of keratinous fibers using the cosmetic agents are provided herein. In one embodiment, the cosmetic agent for the temporary deformation of keratinous fibe includes a cosmetic preparation. The cosmetic preparation contains at least one copolymer a1) composed at least of the following monomer units: styrene, and at least one of acrylic acid or methacrylic acid. The cosmetic preparation further contains at least one copolymer a2) composed at least of the following monomer units: N-tert-butylacrylamide, acrylic acid, and ethyl acrylate. The weight fraction of copolymers a1) and a2) in the total weight of the cosmetic preparation is about 10 to about 30% by weight.

In another embodiment, a cosmetic product including a cosmetic agent and a dispensing device with a spray valve is provided. The cosmetic agent includes at least one copolymer a1) composed at least of the following monomer units: styrene, and at least one of acrylic acid or methacrylic acid. The cosmetic agent further includes at least one copolymer a2) composed at least of the following monomer units: N-tert-butylacrylamide, acrylic acid, and ethyl acrylate. The weight fraction of copolymers a1) and a2) in the total weight of the cosmetic agent is about 10 to about 30% by weight.

In another embodiment, the method for the temporary deformation of keratinous fibers includes the step of acting on the keratinous fibers by a cosmetic agent. The cosmetic agent includes at least one copolymer a1) composed at least of the following monomer units: styrene, and at least one of acrylic acid or methacrylic acid. The cosmetic agent further includes at least one copolymer a2) composed at least of the following monomer units: N-tert-butylacrylamide, acrylic acid, and ethyl acrylate. The weight fraction of copolymers a1) and a2) in the total weight of the cosmetic agent is about 10 to about 30% by weight. The method further includes the step of temporarily fixing the keratinous fibers into shape.

### DETAILED DESCRIPTION

The following detailed description is merely exemplary in nature and is not intended to limit the disclsoure or the application and uses of the disclosure. Furthermore, there is not intention to be bound by any theory presented in the preceding background or the following detailed description.

It is contemplated herein to provide further suitable polymer combinations which are characterized by good film-forming and/or setting properties and which have a very high degree of hold, without having to forgo flexibility and good moisture resistance, in particular to perspiration, and water resistance. A further aim is that the polymer combinations are suitable for producing cosmetic compositions having high chemical and physical stability, and are easy to apply.

This object is achieved as contemplated herein by a combination of two specific, different copolymers.

The present disclosure provides the following:
1. A cosmetic agent for the temporary deformation of keratinous fibers, comprising
   a) a cosmetic preparation containing
      a1) at least one copolymer composed at least of the following monomer units:
         styrene
         acrylic acid and/or methacrylic acid;
      a2) at least one copolymer composed at least of the following monomer units:
         N-tert-butylacrylamide
         acrylic acid
         ethyl acrylate,
wherein the weight fraction of copolymers a1) and a2) in the total weight of the cosmetic preparation is about 10 to about 30% by weight.
2. The cosmetic agent according to item 1, wherein the weight fraction of copolymers a1) and a2) in the total weight of the cosmetic preparation is about 11 to about 25% by weight and in particular about 12 to about 20% by weight.
3. The cosmetic agent according to one of the preceding items, wherein the at least one copolymer a1), based on its total weight, is composed of at least 90% by weight, preferably at least 95% by weight, and in particular at least 97% by weight of the monomers
   styrene
   acrylic acid and/or methacrylic acid.
4. The cosmetic agent according to one of the preceding items, wherein the at least one copolymer a1), based on its total weight, is composed of at least 90% by weight, preferably at least 95% by weight, and in particular at least 97% by weight of the monomers
   styrene
   acrylic acid and/or methacrylic acid
   acrylic acid ester and/or methacrylic acid ester.
5. The cosmetic agent according to one of the preceding items, wherein copolymer a1) has the INCI name Styrene/Acrylates Copolymer.
6. The cosmetic agent according to one of the preceding items, wherein the preparation, based on its total weight, contains about 1.0 to about 15% by weight, preferably about 2.0 to about 14% by weight, and in particular about 4.0 to about 12% by weight of copolymer a1).
7. The cosmetic agent according to one of the preceding items, wherein the at least one copolymer a2), based on its total weight, is composed of at least 90% by weight, preferably at least 95% by weight, and in particular at least 97% by weight of the monomers
   N-tert-butylacrylamide
   acrylic acid
   ethyl acrylate.

8. The cosmetic agent according to one of the preceding items, wherein copolymer a2) has the INCI name Acrylates/t-Butylacrylamide Copolymer.
9. The cosmetic agent according to one of the preceding items, wherein the preparation, based on its total weight, contains about 1.0 to about 15% by weight, preferably about 2.0 to about 14% by weight, and in particular about 4.0 to about 12% by weight of copolymer a2).
10. The cosmetic agent according to one of the preceding items, wherein the weight ratio of copolymer a1) to copolymer a2) is about 1:7 to about 7:1, preferably about 1:5 to about 5:1, and in particular about 1:3 to about 3:1.
11. The cosmetic agent according to one of the preceding items, wherein the preparation, based on its total weight, contains about 30 to about 90% by weight, preferably about 40 to about 85% by weight, and in particular about 50 to about 80% by weight of polar solvent.
12. The cosmetic agent according to one of the preceding items, wherein the preparation, based on its total weight, contains about 0.01 to about 30% by weight, in particular about 5.0 to about 25% by weight of water.
13. The cosmetic agent according to one of the preceding items, wherein the preparation, based on its total weight, is composed of at least 70% by weight, preferably at least 80% by weight, and in particular at least 90% by weight of copolymers a1) and a2), ethanol, and water.
14. The cosmetic agent according to one of the preceding items, wherein preparation a) has a viscosity (Brookfield DV2T viscometer; 20° C., spindle 2, 10 rpm) of about 10 to about 1000 mPas, preferably about 15 to about 500 mPas, and in particular about 20 to about 200 mPas.
15. The cosmetic agent according to one of the preceding items, wherein the agent also includes
   b) at least one propellant.
16. The cosmetic agent according to one of the preceding items, wherein the agent also includes
   b) at least one propellant from the group propane, a mixture of propane and butane, dimethyl ether, and 1,1-difluoroethane.
17. The cosmetic agent according to one of the preceding items which, based on its total weight, includes
   a) about 30 to about 60% by weight of the cosmetic preparation
   b) about 40 to about 70% by weight of propellant.
18. A cosmetic product comprising
   i) a cosmetic agent according to one of items 1 to 17
   ii) a dispensing device having a spray valve.
19. The cosmetic agent according to item 18, wherein the spray valve has a maximum valve opening less than 0.4 mm, preferably between about 0.22 and about 0.32 mm, and in particular between about 0.25 und about 0.30 mm.
20. Use of an agent or a product according to one of items 1 to 21 for the temporary deformation of keratin-containing fibers, in particular human hair.
21. A method for the temporary deformation of keratin-containing fibers, in particular human hair, in which the keratinous fibers are acted on by a cosmetic agent according to one of items 1 to 17 and temporarily fixed in shape.
22. The method according to item 21, wherein the action of the keratinous fibers occurs at a spray rate of the cosmetic preparation of about 18 to about 30 g/min, preferably about 22 to about 28 g/min.

It has surprisingly been found within the scope of the present disclosure that improved moisture resistance of styling products may be obtained by combining two components, known per se, already used in styling products. Other properties of styling products which are customarily required, such as long-term hold, stiffness, and low tack, are maintained. Such a good combination of properties was not to be expected, even with knowledge of the individual components, and was surprising. It has been shown experimentally that a highly superadditive, i.e., synergistic, effect with regard to moisture resistance and the degree of hold has been obtained by the combination of the two components.

As contemplated herein, the term “keratinous fibers” includes fur, wool, and feathers, but in particular human hair.

The essential components of the cosmetic composition as contemplated herein are the anionic copolymer a1) and the anionic copolymer a2), which is different from copolymer a1).

The cosmetic preparations as contemplated herein contain an anionic copolymer a1) as the first essential component.

With regard to the manufacturability, applicability, and cosmetic effect of cosmetic agents as contemplated herein, it has proven advantageous when the weight fraction of copolymer a1) in the total weight of cosmetic preparation a) is about 1.0 to about 15% by weight, preferably about 2.0 to about 14% by weight, and in particular about 4.0 to about 12% by weight.

Copolymer a1) may be based on the monomers i) styrene and ii) acrylic acid and/or methacrylic acid, and optionally further monomers.

Preferred copolymers a1) are preferably composed of at least 90% by weight, preferably at least 95% by weight, and in particular at least 97% by weight of the monomers styrene and acrylic acid and/or methacrylic acid. Particularly preferred copolymers a) have been obtained solely from the monomers styrene and acrylic acid and/or methacrylic acid.

The cosmetic agents in another preferred embodiment are characterized in that the at least one copolymer a1), based on its total weight, is composed of at least 90% by weight, preferably at least 95% by weight, and in particular at least 97% by weight of the monomers
styrene
acrylic acid and/or methacrylic acid
acrylic acid ester and/or methacrylic acid ester.

The above-described copolymers a1) are marketed, for example, by Dow Chemicals under the name Acudyne® Shine (INCI name: Styrene/Acrylates Copolymer; CAS No. 9010-92-8).

Copolymer a1) is preferably used in the cosmetic preparation in partially neutralized or neutralized form. At least one alkanolamine is preferably used for the neutralization. The alkanolamines which are usable as alkalizing agent as contemplated herein are preferably selected from primary amines having a C₂-C₆ alkyl base structure bearing at least one hydroxyl group. Particularly preferred alkanolamines are selected from the group comprising 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol. Alkanolamines very particularly preferred as contemplated herein are selected from the group 2-aminoethan-1-ol, 2-amino-2-methylpropan-1-ol, and 2-amino-2-methylpropane-1,3-diol. 2-Amino-2-methylpropanol has proven to be a particularly suitable neutralizing agent. Cosmetic agents preferred as contemplated herein contain at least one alkanolamine, preferably 2-amino-2-methylpropanol. The 2-amino-2-methylpropanol is preferably used in the cosmetic preparations as contemplated herein in a quantity which does not exceed the quantity necessary for neutralizing the copolymer a1). The quantities of 2-amino-2-methylpropanol used in the cosmetic preparations as contemplated herein are preferably about 80 to 100%, particularly preferably about 90 to 100%, and in particular about 95 to 100% of the quantity necessary for completely neutralizing the copolymer a1). In one preferred embodiment, the weight fraction of 2-amino-2-methylpropanol in the total weight of cosmetic preparation a) is about 0.1 to about 4.0% by weight, preferably about 0.5 to about 3.0% by weight, and in particular about 1.0 to about 2.0% by weight.

The cosmetic preparations as contemplated herein contain an anionic acrylate copolymer a2) as the second essential component.

With regard to the manufacturability, applicability, and cosmetic effect of cosmetic agents as contemplated herein, it has proven advantageous when the weight fraction of copolymer a2) in the total weight of cosmetic preparation a) is about 1.0 to about 15% by weight, preferably about 2.0 to about 14% by weight, and in particular about 4.0 to about 12% by weight.

Copolymer a2) may be based on the monomers N-tert-butylacrylamide, acrylic acid, ethyl acrylate, and optionally further monomers.

Preferred copolymers a2) are preferably composed of at least 90% by weight, preferably at least 95% by weight, and in particular at least 97% by weight of the monomers i) N-tert-butylacrylamide, ii) acrylic acid, iii) ethyl acrylate. Particularly preferred copolymers a2) have been obtained solely from the monomers i) N-tert-butylacrylamide, ii) acrylic acid, iii) ethyl acrylate.

The above-described copolymers a2) are marketed, for example, by BASF under the name Ultrahold® 8 (INCI name: Acrylates/t-Butylacrylamide Copolymer; CAS No. 26062-56-6).

Copolymer a2) is preferably used in the cosmetic agents in partially neutralized or neutralized form. At least one alkanolamine is preferably used for the neutralization. The alkanolamines which are usable as alkalizing agent as contemplated herein are preferably selected from primary amines having a C₂-C₆ alkyl base structure bearing at least one hydroxyl group. Particularly preferred alkanolamines are selected from the group comprising 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, 2-and amino-2-methylpropane-1,3-diol. Alkanolamines very particularly preferred as contemplated herein are selected from the group 2-aminoethan-1-ol, 2-amino-2-methylpropan-1-ol, and 2-amino-2-methylpropane-1,3-diol. 2-Amino-2-methylpropanol has proven to be a particularly suitable neutralizing agent. Cosmetic agents preferred as contemplated herein contain at least one alkanolamine, preferably 2-amino-2-methylpropanol. The 2-amino-2-methylpropanol is preferably used in the cosmetic agents as contemplated herein in a quantity which does not exceed the quantity necessary for neutralizing the copolymer a2). The quantities of 2-amino-2-methylpropanol used in the cosmetic preparations as contemplated herein are preferably about 80 to 100%, particularly preferably about 90 to 100%, and in particular about 95 to 100% of the quantity necessary for completely neutralizing the copolymer a2). In one preferred embodiment, the weight fraction of 2-amino-2-methylpropanol in the total weight of the cosmetic preparation a) is about 0.1 to about 4.0% by weight, preferably about 0.5 to about 3.0% by weight, and in particular about 1.0 to about 2.0% by weight.

The weight fraction of copolymers a1) and a2) in the total weight of the cosmetic preparation is about 10 to about 30% by weight. Cosmetic preparations are preferred in which the weight fraction of copolymers a1) and a2) in the total weight of the cosmetic preparation is about 11 to about 25% by weight, in particular about 12 to about 20% by weight.

In addition to the total weight fraction of copolymers a1) and a2), the weight ratio of copolymers a1) and a2) with respect to one another also has an influence on the moisture resistance, the degree of hold, and the other application properties of cosmetic agents as contemplated herein. Cosmetic agents that are particularly advantageous technically are characterized in that the weight ratio of copolymer a1) to copolymer a2) is about 1:7 to about 7:1, preferably about 1:5 to about 5:1, and in particular about 1:3 to about 3:1.

In addition to the above-described copolymers a1) and copolymers a2), the cosmetic preparations as contemplated herein may contain further active substances, auxiliary substances, and care substances.

Film-forming polymers constitute a first group of preferably used active substances. These film-forming polymers are not identical to copolymer a1) or copolymer a2) described above. The weight fraction of the film-forming polymer in the total weight of the cosmetic preparation is preferably about 0.1 to about 8.0% by weight, preferably about 0.5 to about 6.0% by weight, and in particular about 1.0 to about 4.0% by weight.

Nonionic polymers are particularly preferably used as film-forming polymers. Examples of suitable nonionic polymers include the following:
vinylpyrrolidone/vinyl ester copolymers as marketed, for example, under the trade name Luviskol® (BASF). Luviskol® VA 64 and Luviskol® VA 73, each of which are vinylpyrrolidone/vinyl acetate copolymers, are preferred nonionic polymers.
cellulose ethers such as hydroxypropylcellulose, hydroxyethylcellulose, and methylhydroxypropylcellulose as marketed, for example, under the trade names Culminal® and Benecel® (Aqualon).
shellac.
polyvinylpyrrolidones as marketed, for example, under the name Luviskol® (BASF).
siloxanes. These siloxanes may be water-soluble as well as nonwater-soluble. Volatile as well as nonvolatile siloxanes are suitable; nonvolatile siloxanes are understood to mean those compounds whose boiling point at standard pressure is above 200° C. Preferred siloxanes are polydialkylsiloxanes, for example polydimethylsiloxane, polyalkylarylsiloxanes, for example polyphenylmethylsiloxane, ethoxylated polydialkylsiloxanes, and polydialkylsiloxanes which contain amine and/or hydroxy groups.
glycosidically substituted silicones.

Due to their cosmetic effect in combination with copolymers a1) and a2), film-forming polymers preferably used as contemplated herein are in particular polyvinylpyrrolidones (INCI name: PVP) and vinylpyrrolidone/vinyl acetate copolymers (INCI name: VP/VA Copolymer), wherein the weight fraction of these polymers is preferably limited to quantities between about 1.0 and about 10% by weight. Particularly preferred cosmetic preparations as contemplated herein are therefore characterized in that, based on their total weight, they also contain about 1.0 to about 10% by weight polyvinylpyrrolidone and/or vinylpyrrolidone/vinyl acetate copolymer, preferably polyvinylpyrrolidone. Particularly preferred cosmetic preparations have a weight fraction of polyvinylpyrrolidone and/or vinylpyrrolidone/vinyl acetate copolymer in the total weight of the cosmetic preparation of about 2.0 to about 8.5% by weight, preferably about 3.0 to about 7.0% by weight.

In summary, cosmetic agents particularly preferred as contemplated herein, including copolymers a1) and a2) and the film-forming polymer a3), contain three different polymers.

Protein hydrolysates and/or the derivatives thereof may be used as care substance. Protein hydrolysates are product mixtures that are obtained by acidically, basically, or enzymatically catalyzed degradation of proteins. As contemplated herein, the term “protein hydrolysates” is also understood to total hydrolysates and individual amino acids and the derivatives thereof, as well as mixtures of various amino acids. The molar weight of the protein hydrolysates that are usable as contemplated herein is between 75 (the molar weight of glycine) and 200,000 Dalton; the molar weight is preferably about 75 to about 50,000 Dalton, and very particularly about 75 to about 20,000 Dalton.

Vitamins, provitamins, vitamin precursors, and/or the derivatives thereof constitute another group of care substances. As contemplated herein, vitamins, provitamins and vitamin precursors generally associated with the groups A, B, C, E, F, and H are preferred.

Further care substances are glycerin, propylene glycol, panthenol, caffeine, nicotinamide, and sorbitol.

Plant extract and also mono- and oligosaccharides and/or lipids may be used as care substance.

The compositions of several cosmetic preparations a) used, in which the weight fraction of copolymers a1) and a2) in the total weight of the cosmetic preparation is about 10 to about 30% by weight, preferably about 11 to about 25% by weight, and in particular about 12 to about 20% by weight, may be found in the following tables. (Unless stated otherwise, the indications in % by weight refer to the total weight of the cosmetic agent.)

| | Formula 1 | Formula 2 | Formula 3 | Formula 4 | Formula 5 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Optional additives | to make 100 | to make 100 | to make 100 | to make 100 | to make 100 |
| | Formula 6 | Formula 7 | Formula 8 | Formula 9 | Formula 10 |
| Styrene/Acrylates | 1.0 to 15 | 1.5 to 14.5 | 2.0 to 14 | 3.0 to 13 | 4.0 to 12 |
| Copolymer (INCI) | | | | | |
| Copolymer a2) * | 1.0 to 15 | 1.5 to 14.5 | 2.0 to 14 | 3.0 to 13 | 4.0 to 12 |
| Optional additives | to make 100 | to make 100 | to make 100 | to make 100 | to make 100 |
| | Formula 11 | Formula 12 | Formula 13 | Formula 14 | Formula 15 |
| Copolymer a1) * | 1.0 to 15 | 1.5 to 14.5 | 2.0 to 14 | 3.0 to 13 | 4.0 to 12 |
| Acrylates/t- | 1.0 to 15 | 1.5 to 14.5 | 2.0 to 14 | 3.0 to 13 | 4.0 to 12 |
| Butylacrylamide | | | | | |
| Copolymer (INCI) | | | | | |
| Optional additives | to make 100 | to make 100 | to make 100 | to make 100 | to make 100 |
| | Formula 16 | Formula 17 | Formula 18 | Formula 19 | Formula 20 |
| Styrene/Acrylates | 1.0 to 15 | 1.5 to 14.5 | 2.0 to 14 | 3.0 to 13 | 4.0 to 12 |
| Copolymer (INCI) | | | | | |
| Acrylates/t- | 1.0 to 15 | 1.5 to 14.5 | 2.0 to 14 | 3.0 to 13 | 4.0 to 12 |
| Butylacrylamide | | | | | |
| Copolymer (INCI) | | | | | |
| Optional additives | to make 100 | to make 100 | to make 100 | to make 100 | to make 100 |

* according to Claim 1

Preferred cosmetic agents are based on an aqueous, aqueous/alcoholic, or alcoholic carrier. Thus, preferred cosmetic agents, based on their total weight, contain about 40 to about 98% by weight, preferably about 60 to about 95% by weight, and in particular about 70 to about 92% by weight of polar solvent, preferably polar solvent from the group water, ethanol, and isopropanol.

As previously mentioned, the lower alcohols having 1 to 4 carbon atoms, for example ethanol and isopropanol, customarily used for cosmetic purposes may be contained as alcohols.

In addition to these alcoholic solvents, water-soluble cosolvents, in particular in combination with water, are also usable. Examples of particularly preferred cosolvents are glycerin and/or ethylene glycol and/or 1,2-propylene glycol, which are preferably used in a quantity of 0 to about 30% by weight, based on cosmetic preparation a).

Together with copolymers a1) and a2) described above, the aqueous, aqueous/alcoholic, or alcoholic carriers preferably form an essential component of cosmetic preparations a) as contemplated herein. Particularly preferred are cosmetic preparations which, based on their total weight, are composed of at least 70% by weight, preferably at least 80% by weight, and in particular at least 90% by weight of copolymers a1) and a2), ethanol, and/or water.

The composition of several technically advantageous cosmetic preparations a) having a liquid carrier, in which the weight fraction of copolymers a1) and a2) in the total weight of the cosmetic preparation is about 1.0 to about 10% by weight, preferably about 1.5 to about 9.0% by weight, and in particular about 2.0 to about 8.0% by weight, may be found in the following tables. (Unless stated otherwise, the indications in % by weight refer to the total weight of the cosmetic agent.)

| | Formula 21 | Formula 22 | Formula 23 | Formula 24 | Formula 25 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| Optional additives | to make 100 | to make 100 | to make 100 | to make 100 | to make 100 |
| | Formula 26 | Formula 27 | Formula 28 | Formula 29 | Formula 30 |
| Styrene/Acrylates | 1.0 to 15 | 1.5 to 14.5 | 2.0 to 14 | 3.0 to 13 | 4.0 to 12 |
| Copolymer (INCI) | | | | | |
| Copolymer a2) * | 1.0 to 15 | 1.5 to 14.5 | 2.0 to 14 | 3.0 to 13 | 4.0 to 12 |
| Water and/or ethanol | 40 to 98 | 40 to 98 | 60 to 95 | 60 to 95 | 70 to 92 |
| Optional additives | to make 100 | to make 100 | to make 100 | to make 100 | to make 100 |
| | Formula 31 | Formula 32 | Formula 33 | Formula 34 | Formula 35 |
| Copolymer a1) * | 1.0 to 15 | 1.5 to 14.5 | 2.0 to 14 | 3.0 to 13 | 4.0 to 12 |
| Acrylates/t- | 1.0 to 15 | 1.5 to 14.5 | 2.0 to 14 | 3.0 to 13 | 4.0 to 12 |
| Butylacrylamide | | | | | |
| Copolymer (INCI) | | | | | |
| Water and/or ethanol | 40 to 98 | 40 to 98 | 60 to 95 | 60 to 95 | 70 to 92 |
| Optional additives | to make 100 | to make 100 | to make 100 | to make 100 | to make 100 |
| | Formula 36 | Formula 37 | Formula 38 | Formula 39 | Formula 40 |
| Styrene/Acrylates | 1.0 to 15 | 1.5 to 14.5 | 2.0 to 14 | 3.0 to 13 | 4.0 to 12 |
| Copolymer (INCI) | | | | | |
| Acrylates/t- | 1.0 to 15 | 1.5 to 14.5 | 2.0 to 14 | 3.0 to 13 | 4.0 to 12 |
| Butylacrylamide | | | | | |
| Copolymer (INCI) | | | | | |
| Water and/or ethanol | 40 to 98 | 40 to 98 | 60 to 95 | 60 to 95 | 70 to 92 |
| Optional additives | to make 100 | to make 100 | to make 100 | to make 100 | to make 100 |

* according to Claim 1

Of course, it is possible not only to vary the weight fraction of the liquid carrier in the total weight of the cosmetic preparation a), but also to change the weight ratio of aqueous to alcoholic carrier.

Preferred cosmetic preparations contain about 0.01 to about 30% by weight, in particular about 5.0 to about 25% by weight of water, based on their total weight.

As previously mentioned, in particular the lower alcohols having 1 to 4 carbon atoms, customarily used for cosmetic purposes, for example ethanol and isopropanol, may be used as alcohols. Preferred cosmetic preparations contain about 30 to about 90% by weight, preferably about 40 to about 85% by weight, and in particular about 50 to about 80% by weight of ethanol, based on their total weight.

The composition of several technically advantageous cosmetic preparations a) having a liquid carrier, in which the weight fraction of copolymers a1) and a2) in the total weight of the cosmetic preparation is about 10 to about 30% by weight, preferably about 11 to about 25% by weight, and in particular 12 to 20% by weight, may be found in the following tables. (Unless stated otherwise, the indications in % by weight refer to the total weight of the cosmetic agent.)

| | Formula 41 | Formula 42 | Formula 43 | Formula 44 | Formula 45 |
|---|---|---|---|---|---|
| Copolymer a1) * | 1.0 to 15 | 1.5 to 14.5 | 2.0 to 14 | 3.0 to 13 | 4.0 to 12 |
| Copolymer a2) * | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | to make 100 | to make 100 | to make 100 | to make 100 | to make 100 |
| | Formula 46 | Formula 47 | Formula 48 | Formula 49 | Formula 50 |
| Styrene/Acrylates | 1.0 to 15 | 1.5 to 14.5 | 2.0 to 14 | 3.0 to 13 | 4.0 to 12 |
| Copolymer (INCI) | | | | | |
| Copolymer a2) * | 1.0 to 15 | 1.5 to 14.5 | 2.0 to 14 | 3.0 to 13 | 4.0 to 12 |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | to make 100 | to make 100 | to make 100 | to make 100 | to make 100 |
| | Formula 51 | Formula 52 | Formula 53 | Formula 54 | Formula 55 |
| Copolymer a1) * | 1.0 to 15 | 1.5 to 14.5 | 2.0 to 14 | 3.0 to 13 | 4.0 to 12 |
| Acrylates/t- | 1.0 to 15 | 1.5 to 14.5 | 2.0 to 14 | 3.0 to 13 | 4.0 to 12 |
| butylacrylamide | | | | | |
| Copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | to make 100 | to make 100 | to make 100 | to make 100 | to make 100 |
| | Formula 56 | Formula 57 | Formula 58 | Formula 59 | Formula 60 |
| Styrene/Acrylates | 1.0 to 15 | 1.5 to 14.5 | 2.0 to 14 | 3.0 to 13 | 4.0 to 12 |
| Copolymer (INCI) | | | | | |
| Acrylates/t- | 1.0 to 15 | 1.5 to 14.5 | 2.0 to 14 | 3.0 to 13 | 4.0 to 12 |
| Butylacrylamide | | | | | |
| Copolymer (INCI) | | | | | |
| Ethanol | 30 to 90 | 30 to 90 | 40 to 85 | 40 to 85 | 50 to 80 |
| Water | 0.01 to 30 | 0.1 to 30 | 1.0 to 30 | 2.0 to 30 | 5.0 to 25 |
| Optional additives | to make 100 | to make 100 | to make 100 | to make 100 | to make 100 |

* according to Claim 1

The cosmetic preparation is preferably is sprayed on the hair. This is particularly preferably carried out using a propellant (aerosol spray). Preferred cosmetic agents therefore include at least one propellant b) in addition to the cosmetic preparation a).

Suitable propellants (propellant gases) are propane, propene, n-butane, isobutane, isobutene, n-pentane, pentene, isopentane, isopentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, individually and also in combination. Hydrophilic propellant gases such as carbon dioxide may also be advantageously used within the meaning of the present disclosure when the proportion of hydrophilic gases is selected to be low, and lipophilic propellant gas (propane/butane, for example) is present in excess. Propane, n-butane, isobutane, and mixtures of these propellant gases are particularly preferred. Preferred cosmetic agents are characterized in that the agent also includes at least one propellant b) from the group propane, a mixture of propane and butane, dimethyl ether, and 1,1-difluoroethane (INCI: Hydrofluorocarbon 152a).

Preferred cosmetic agents as contemplated herein contain 30 to 60% by weight of cosmetic preparation a) and about 40 to about 70% by weight of propellant b), based on their total weight.

The further composition of several preferred cosmetic agents which in addition to cosmetic preparation a) also include a propellant b), and in which the weight fraction of copolymers a1) and a2) in the total weight of the cosmetic preparation is about 10 to about 30% by weight, preferably about 11 to about 25% by weight, and in particular about 12 to about 20% by weight, may be found in Table 1 below.

In Table 1, the left column (“Formula x”) refers in each case to one of the cosmetic preparations a) of formulas 1 to 60 by way of example, listed in the tables presented above. Columns two through five (“propellant”) in each case indicate the quantity of propellant that is combined with the corresponding cosmetic preparation. These indications in “% by weight” refer to the total weight of cosmetic preparation a) having the particular “Formula x,” without propellant.

The indication “50 to 200% by weight” in Table 1 below corresponds to the addition of propellant to cosmetic preparation a) in a quantity that is about 50 to about 200% by weight of the weight of cosmetic preparation a). In other words, the cosmetic preparation a) and the propellant b) are present in this cosmetic agent in a weight ratio of about 100:50 to about 100:200 and about 2:1 to about 1:2, respectively.

Accordingly, in the cosmetic agents in line 4, column 4 of Table 1 below, a mixture of the propellant-free cosmetic preparation a) according to formula 3 with a propane/butane mixture, in a weight ratio of cosmetic preparation a) to propellant about 100:50 to about 100:200, is described. In other words, the entry in line 4, column 4 describes a cosmetic agent for the temporary deformation of keratinous fibers, comprising
a) a cosmetic preparation containing
   a1) about 2.0 to about 14% by weight of at least one copolymer composed at least of the following monomer units:
      styrene
      acrylic acid and/or methacrylic acid;
   a2) about 2.0 to about 14% by weight of at least one copolymer composed at least of the following monomer units:
      N-tert-butylacrylamide
      acrylic acid
      ethyl acrylate,
b) propellant from the group of propane/butane mixtures,
   wherein the weight fraction of copolymers a1) and a2) in the total weight of the cosmetic preparation is about 10 to about 30% by weight, and the weight ratio of cosmetic preparation a) to propellant b) is about 2:1 to about 1:2.

TABLE 1

| | Propellant [% by weight] | | | |
|---|---|---|---|---|
| Formula 1 | 50 to 200 | 50 to 200 DFE* | 50 to 200 P/B** | 50 to 200 |
| | | | | DME*** |
| Formula 2 | 50 to 200 | 50 to 200 DFE* | 50 to 200 P/B** | 50 to 200 |
| | | | | DME*** |
| Formula 3 | 50 to 200 | 50 to 200 DFE* | 50 to 200 P/B** | 50 to 200 |
| | | | | DME*** |
| Formula 4 | 50 to 200 | 50 to 200 DFE* | 50 to 200 P/B** | 50 to 200 |
| | | | | DME*** |
| Formula 5 | 50 to 200 | 50 to 200 DFE* | 50 to 200 P/B** | 50 to 200 |
| | | | | DME*** |
| Formula 6 | 50 to 200 | 50 to 200 DFE* | 50 to 200 P/B** | 50 to 200 |
| | | | | DME*** |
| Formula 7 | 50 to 200 | 50 to 200 DFE* | 50 to 200 P/B** | 50 to 200 |
| | | | | DME*** |
| Formula 8 | 50 to 200 | 50 to 200 DFE* | 50 to 200 P/B** | 50 to 200 |
| | | | | DME*** |
| Formula 9 | 50 to 200 | 50 to 200 DFE* | 50 to 200 P/B** | 50 to 200 |
| | | | | DME*** |
| Formula 10 | 50 to 200 | 50 to 200 DFE* | 50 to 200 P/B** | 50 to 200 |
| | | | | DME*** |
| Formula 11 | 50 to 200 | 50 to 200 DFE* | 50 to 200 P/B** | 50 to 200 |
| | | | | DME*** |
| Formula 12 | 50 to 200 | 50 to 200 DFE* | 50 to 200 P/B** | 50 to 200 |
| | | | | DME*** |
| Formula 13 | 50 to 200 | 50 to 200 DFE* | 50 to 200 P/B** | 50 to 200 |
| | | | | DME*** |
| Formula 14 | 50 to 200 | 50 to 200 DFE* | 50 to 200 P/B** | 50 to 200 |
| | | | | DME*** |
| Formula 15 | 50 to 200 | 50 to 200 DFE* | 50 to 200 P/B** | 50 to 200 |
| | | | | DME*** |
| Formula 16 | 50 to 200 | 50 to 200 DFE* | 50 to 200 P/B** | 50 to 200 |
| | | | | DME*** |
| Formula 17 | 50 to 200 | 50 to 200 DFE* | 50 to 200 P/B** | 50 to 200 |
| | | | | DME*** |
| Formula 18 | 50 to 200 | 50 to 200 DFE* | 50 to 200 P/B** | 50 to 200 |
| | | | | DME*** |
| Formula 19 | 50 to 200 | 50 to 200 DFE* | 50 to 200 P/B** | 50 to 200 |
| | | | | DME*** |
| Formula 20 | 50 to 200 | 50 to 200 DFE* | 50 to 200 P/B** | 50 to 200 |
| | | | | DME*** |
| Formula 21 | 50 to 200 | 50 to 200 DFE* | 50 to 200 P/B** | 50 to 200 |
| | | | | DME*** |
| Formula 22 | 50 to 200 | 50 to 200 DFE* | 50 to 200 P/B** | 50 to 200 |
| | | | | DME*** |
| Formula 23 | 50 to 200 | 50 to 200 DFE* | 50 to 200 P/B** | 50 to 200 |
| | | | | DME*** |
| Formula 24 | 50 to 200 | 50 to 200 DFE* | 50 to 200 P/B** | 50 to 200 |
| | | | | DME*** |
| Formula 25 | 50 to 200 | 50 to 200 DFE* | 50 to 200 P/B** | 50 to 200 |
| | | | | DME*** |
| Formula 26 | 50 to 200 | 50 to 200 DFE* | 50 to 200 P/B** | 50 to 200 |
| | | | | DME*** |
| Formula 27 | 50 to 200 | 50 to 200 DFE* | 50 to 200 P/B** | 50 to 200 |
| | | | | DME*** |
| Formula 28 | 50 to 200 | 50 to 200 DFE* | 50 to 200 P/B** | 50 to 200 |
| | | | | DME*** |
| Formula 29 | 50 to 200 | 50 to 200 DFE* | 50 to 200 P/B** | 50 to 200 |
| | | | | DME*** |
| Formula 30 | 50 to 200 | 50 to 200 DFE* | 50 to 200 P/B** | 50 to 200 |
| | | | | DME*** |
| Formula 31 | 50 to 200 | 50 to 200 DFE* | 50 to 200 P/B** | 50 to 200 |
| | | | | DME*** |
| Formula 32 | 50 to 200 | 50 to 200 DFE* | 50 to 200 P/B** | 50 to 200 |
| | | | | DME*** |
| Formula 33 | 50 to 200 | 50 to 200 DFE* | 50 to 200 P/B** | 50 to 200 |
| | | | | DME*** |
| Formula 34 | 50 to 200 | 50 to 200 DFE* | 50 to 200 P/B** | 50 to 200 |
| | | | | DME*** |
| Formula 35 | 50 to 200 | 50 to 200 DFE* | 50 to 200 P/B** | 50 to 200 |
| | | | | DME*** |
| Formula 36 | 50 to 200 | 50 to 200 DFE* | 50 to 200 P/B** | 50 to 200 |
| | | | | DME*** |
| Formula 37 | 50 to 200 | 50 to 200 DFE* | 50 to 200 P/B** | 50 to 200 |
| | | | | DME*** |
| Formula 38 | 50 to 200 | 50 to 200 DFE* | 50 to 200 P/B** | 50 to 200 |
| | | | | DME*** |
| Formula 39 | 50 to 200 | 50 to 200 DFE* | 50 to 200 P/B** | 50 to 200 |
| | | | | DME*** |
| Formula 40 | 50 to 200 | 50 to 200 DFE* | 50 to 200 P/B** | 50 to 200 |
| | | | | DME*** |
| Formula 41 | 50 to 200 | 50 to 200 DFE* | 50 to 200 P/B** | 50 to 200 |
| | | | | DME*** |
| Formula 42 | 50 to 200 | 50 to 200 DFE* | 50 to 200 P/B** | 50 to 200 |
| | | | | DME*** |
| Formula 43 | 50 to 200 | 50 to 200 DFE* | 50 to 200 P/B** | 50 to 200 |
| | | | | DME*** |
| Formula 44 | 50 to 200 | 50 to 200 DFE* | 50 to 200 P/B** | 50 to 200 |
| | | | | DME*** |
| Formula 45 | 50 to 200 | 50 to 200 DFE* | 50 to 200 P/B** | 50 to 200 |
| | | | | DME*** |
| Formula 46 | 50 to 200 | 50 to 200 DFE* | 50 to 200 P/B** | 50 to 200 |
| | | | | DME*** |
| Formula 47 | 50 to 200 | 50 to 200 DFE* | 50 to 200 P/B** | 50 to 200 |
| | | | | DME*** |
| Formula 48 | 50 to 200 | 50 to 200 DFE* | 50 to 200 P/B** | 50 to 200 |
| | | | | DME*** |
| Formula 49 | 50 to 200 | 50 to 200 DFE* | 50 to 200 P/B** | 50 to 200 |
| | | | | DME*** |
| Formula 50 | 50 to 200 | 50 to 200 DFE* | 50 to 200 P/B** | 50 to 200 |
| | | | | DME*** |
| Formula 51 | 50 to 200 | 50 to 200 DFE* | 50 to 200 P/B** | 50 to 200 |
| | | | | DME*** |
| Formula 52 | 50 to 200 | 50 to 200 DFE* | 50 to 200 P/B** | 50 to 200 |
| | | | | DME*** |
| Formula 53 | 50 to 200 | 50 to 200 DFE* | 50 to 200 P/B** | 50 to 200 |
| | | | | DME*** |
| Formula 54 | 50 to 200 | 50 to 200 DFE* | 50 to 200 P/B** | 50 to 200 |
| | | | | DME*** |
| Formula 55 | 50 to 200 | 50 to 200 DFE* | 50 to 200 P/B** | 50 to 200 |
| | | | | DME*** |
| Formula 56 | 50 to 200 | 50 to 200 DFE* | 50 to 200 P/B** | 50 to 200 |
| | | | | DME*** |
| Formula 57 | 50 to 200 | 50 to 200 DFE* | 50 to 200 P/B** | 50 to 200 |
| | | | | DME*** |
| Formula 58 | 50 to 200 | 50 to 200 DFE* | 50 to 200 P/B** | 50 to 200 |
| | | | | DME*** |
| Formula 59 | 50 to 200 | 50 to 200 DFE* | 50 to 200 P/B** | 50 to 200 |
| | | | | DME*** |
| Formula 60 | 50 to 200 | 50 to 200 DFE* | 50 to 200 P/B** | 50 to 200 |
| | | | | DME*** |

*“DFE” corresponds to 1,1-difluoroethane
**“P/B” corresponds to a propane/butane mixture
***“DME” corresponds to dimethyl ether

Vessels made of metal (aluminum, tinplate, tin), safety or shatterproof plastic, or glass externally coated with plastic, for which pressure resistance and breaking strength, corrosion resistance, ease of filling, as well as esthetic considerations, ease of handling, printability, etc., play a role are suitable as pressurized gas containers for aerosol applications. Special interior protective lacquers ensure corrosion resistance with respect to cosmetic agent a).

If the agents as contemplated herein are to be sprayed on the hair, these agents are advantageously provided with a dispensing device and a spray valve. Accordingly, the resulting cosmetic products include a cosmetic agent as contemplated herein and a dispensing device having a spray valve. Spray valves are preferred which have a maximum valve opening less than 0.4 mm, preferably between about 0.22 and about 0.32 mm, and in particular between about 0.25 and about 0.30 mm. The valve opening surface area is preferably about 60×10−3 to about 100×10−3 mm2.

The spray rate is determined not only by the pressure within the pressurized gas container and the valve opening, but also by the viscosity of cosmetic preparation a). Preferred cosmetic agents are characterized in that preparation a) has a viscosity (Brookfield DV2T viscometer; 20° C., spindle 2, 10 rpm) of about 10 to about 1000 mPas, preferably about 15 to about 500 mPas, and in particular about 20 to about 200 mPas.

In one preferred embodiment of the disclosure, the valve has a valve cone that is coated with a lacquer or a polymeric plastic A, and a similar flexible element with a rebound characteristic such that the valve returns to the closed position (neutral position of the valve) after the actuation is completed. Corresponding cosmetic products are preferred as contemplated herein in which the aerosol dispensing device includes a valve which has a valve cone and/or a flexible element with a rebound characteristic, which are/is coated with a lacquer or a polymeric plastic A.

In another preferred embodiment of the disclosure, the valve has a flexible element with a rebound characteristic and/or a valve cone made of at least one plastic B, preferably an elastomeric plastic. Here as well, cosmetic products as contemplated herein are preferred in which the valve has a flexible element with a rebound characteristic and/or a valve cone made of at least one plastic B, wherein preferred plastics B are elastomeric plastics. Particularly preferred elastomeric plastics are selected from Buna, in particular Buna N, Buna 421, Buna 1602, and Buna KA 6712, neoprene, butyl, and chlorobutyl.

In another preferred embodiment of the disclosure, the flexible element with a rebound characteristic may be designed as a coil spring or helical compression spring. In another preferred embodiment of the disclosure, the flexible element with a rebound characteristic may have a one-piece design with the valve cone and may have flexible legs.

As stated at the outset, the cosmetic agents described above are characterized by special cosmetic properties for the hair, in particular advantageous properties for temporary deformation of hair. A second subject matter of the present patent application is therefore the use of an agent as contemplated herein for the temporary deformation of keratin-containing fibers, in particular human hair.

A third subject matter of the present patent application is a method for the temporary deformation of keratin-containing fibers, in particular human hair, in which the keratinous fibers are acted on by a cosmetic agent as contemplated herein and temporarily fixed in shape.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the invention as set forth in the appended claims.

1. A cosmetic agent for the temporary deformation of keratinous fibers, comprising:
a cosmetic preparation containing:
at least one copolymer a1) composed at least of the following monomer units;
styrene, and
at least one of acrylic acid and/or or methacrylic acid; and
at least one copolymer a2) composed at least of the following monomer units;
N-tert-butylacrylamide,
acrylic acid, and
ethyl acrylate;
wherein the weight fraction of copolymers a1) and a2) in the total weight of the cosmetic preparation is about 10 to about 30% by weight.

2. The cosmetic agent according to claim 1, wherein the weight fraction of copolymers a1) and a2) in the total weight of the cosmetic preparation is about 11 to about 25% by weight.

3. The cosmetic agent according to claim 1,
wherein the preparation, based on its total weight, contains about 1.0 to about 15% by weight of copolymer a1).

4. The cosmetic agent according to claim 1, wherein the preparation, based on its total weight, contains about 1.0 to about 15% by weight, of copolymer a2).

5. The cosmetic agent according to claim 1, wherein the preparation, based on its total weight, contains about 30 to about 90% by weight of ethanol.

6. The cosmetic agent according to claim 1, wherein the preparation, based on its total weight, contains about 0.01 to about 30% by weight of water.

7. The cosmetic agent according to claim 1, wherein the agent also includes at least one propellant.

8. (canceled)

9. (canceled)

10. (canceled)

11. The cosmetic agent according to claim 1, wherein the weight fraction of copolymers a1) and a2) in the total weight of the cosmetic preparation is about 12 to about 20% by weight.

12. The cosmetic agent according to claim 1, wherein the preparation, based on its total weight, contains about 2.0 to about 14% by weight of copolymer a1).

13. The cosmetic agent according to claim 1, wherein the preparation, based on its total weight, contains about 4.0 to about 12% by weight of copolymer a1).

14. The cosmetic agent according to claim 1, wherein the preparation, based on its total weight, contains about 2.0 to about 14% by weight of copolymer a2).

15. The cosmetic agent according to claim 1, wherein the preparation, based on its total weight, contains about 4.0 to about 12% by weight of copolymer a2).

16. The cosmetic agent according to claim 1, wherein the preparation, based on its total weight, contains about 40 to about 85% by weight of ethanol.

17. The cosmetic agent according to claim 1, wherein the preparation, based on its total weight, contains about 50 to about 80% by weight of ethanol.

18. The cosmetic agent according to claim 1, wherein the preparation, based on its total weight, contains about 5 to about 25% by weight of water.

19. The cosmetic agent according to claim 1, wherein copolymer a1) has the INCI name Styrene/Acrylates Copolymer.

20. The cosmetic agent according to claim 1, wherein copolymer a2) has the INCI name Acrylates/t-Butylacrylamide Copolymer.

21. A cosmetic product, comprising:
a cosmetic agent, comprising:
at least one copolymer a1) composed at least of the following monomer units;
styrene, and
at least one of acrylic acid or methacrylic acid; and
at least one copolymer a2) composed at least of the following monomer units;
N-tert-butylacrylamide,
acrylic acid, and
ethyl acrylate;
wherein the weight fraction of copolymers a1) and a2) in the total weight of the cosmetic agent is about 10 to about 30% by weight; and
a dispensing device having a spray valve.

22. A method for the temporary deformation of keratinous fibers, the method comprising the steps of:
acting on the keratinous fibers by a cosmetic agent comprising:
at least one copolymer a1) composed at least of the following monomer units;
styrene, and
at least one of acrylic acid or methacrylic acid; and
at least one copolymer a2) composed at least of the following monomer units;
N-tert-butylacrylamide,
acrylic acid, and
ethyl acrylate;
wherein the weight fraction of copolymers a1) and a2) in the total weight of the cosmetic agent is about 10 to about 30% by weight; and
temporarily fixing the keratinous fibers into shape.
